(19) United States
(12) Patent Application Publication
(10) Pub. No.: US 2013/0041412 A1
(43) Pub. Date: Feb. 14, 2013

(54) FLEXIBLE PEDICLE SCREWS

(71) Applicant: Missoum Moumene, Newton, MA (US)

(72) Inventor: Missoum Moumene, Newton, MA (US)

(73) Assignee: DEPUY SPINE, INC., Raynham, MA (US)

(21) Appl. No.: 13/206,132

(22) Filed: Aug. 9, 2011

(51) Int. Cl. A61B 17/88; A61B 17/86

(52) U.S. Cl. 606/279; 606/309; 606/305

(57) ABSTRACT

Various bone screws and methods for accommodating stiffness regions in bone are provided. The bone screw provided generally includes a receiver member configured to receive a fixation element and an elongate shank having different stiffness regions. In one embodiment, the elongate shank can include at least one slot for increasing the flexibility of the slotted portion of the elongate shank. In another embodiment, the elongate shank can be manufactured from materials selected to alter the stiffness of the shank. The different stiffness regions allow the bone screw to mimic the flexibility of bone, reducing the risk of fracture of the bone and/or loosening of the bone screw.

### FIELD

The present invention relates to flexible bone screws and methods of using the same.

### BACKGROUND

Spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. The fixation rods can have a predetermined contour that has been designed according to the properties of the target implantation site, and once installed, the instrument holds the vertebrae in a desired spatial relationship, either until desired healing or spinal fusion has taken place, or for some longer period of time.

Spinal fixation devices can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a bone screw with a threaded shank that is adapted to be threaded into a vertebra, and a rod-receiving element, usually in the form of a U-shaped head. The shank and rod-receiving element can be provided as a mono axial screw, whereby the rod-receiving element is fixed with respect to the shank, or a polyaxial screw, whereby the rod-receiving element has free angular movement with respect to the shank. In use, the shank portion of each screw is threaded into a vertebra, and once properly positioned, a fixation rod is seated into the rod-receiving element of each screw. The rod is then locked in place by tightening a set-screw, plug, or similar type of fastening mechanism into the rod-receiving element.

Pedicle screws are typically much stiffer than the surrounding bone and more specifically much stiffer than the interior cancellous region of a vertebral body into which the screw is inserted. Motion of the vertebra can cause the screw to undesirably toggle (like a windshield wiper) within the vertebral body, which causes the cancellous bone to fracture. This toggling ultimately leads to screw loosening and failure of the construct. These issues are of particular concern in osteoporotic bone and aging spines.

Accordingly, there remains a need for a pedicle screw that is configured to more closely approximate the flexibility of the different regions of a vertebra.

### SUMMARY

The present invention provides various embodiments of flexible bone screws. In general, a bone screw is provided that includes a receiver member having opposed arms configured to receive a spinal fixation element, and an elongate shank extending distally from the receiver member and having threads formed on its outer surface for engaging bone. The elongate shank can be cannulated or non-cannulated, and include at least one slot extending in a proximal-distal direction. In an exemplary embodiment, the at least one slot is configured to allow flexion of at least a portion of the elongate shank in response to a load applied to the shank when the shank is implanted in bone.

The at least one slot in the elongate shank can have a variety of configurations, and the shank can include any number of slots positioned at various locations. For example, in one exemplary embodiment the elongate shank includes at least one slot on the proximal portion of the shank, and at least one slot on the distal portion of the shank positioned distal of the proximal slot. In another embodiment, the elongate shank can include at least one proximal slot longitudinally aligned and non-continuous with at least one distal slot.

In another embodiment, the elongate shank can include a plurality of slots positioned symmetrically about the shank. In other aspects, the shank can have at least one slot that has a length substantially equal to a proximal-distal length of the shank, and/or that extends in a proximal-distal direction and extends through a central axis of the elongate shank.

In another embodiment, a bone screw is provided having a receiver member with opposed arms configured to receive a spinal fixation element therebetween, and an elongate shaft extending distally from the receiver member and having threads formed on an outer surface thereof for engaging bone. The elongate shank can have at least one slot formed therein and extending in a proximal-distal direction. The at least one slot can be selectively positioned such that, when the elongate shank is disposed within a pedicle of a vertebra, the shank has a flexibility that is configured to mimic a flexibility of the pedicle. In certain aspects, the at least one slot can be positioned to form a first stiffness zone configured to mimic the flexibility of cortical bone and a second stiffness zone configured to mimic the flexibility of cancellous bone. In one embodiment, the at least one slot can include a plurality of slots positioned symmetrically about the shank. In another embodiment, the at least one slot is positioned in the second stiffness zone, while the first stiffness zone is slot-free. In another embodiment, the at least one slot has a length substantially equal to a proximal-distal length of the shank. In yet another embodiment, the at least one slot has opposed openings in opposed outer surfaces of the shank, the opposed openings extending through a central axis of the shank.

The present invention also provides a method for stabilizing bone structures. The method can include advancing a shank of the bone screw into a vertebra such that the threads on the shank threadably engage the vertebra. The shank can have at least one slot that extends in the proximal-distal direction. A spinal fixation rod can be positioned in a rod receiver member that is coupled to the head on the proximal end of the shank of the bone screw, and a fastening element can be applied to the rod receiver member to lock the spinal fixation rod relative to the receiver member. After the fastening element is applied to the rod receiver member, the at least one slot allows the shank to flex in response to a load applied to the shank.

In another embodiment, the at least one slot can extend through the shank such that the slot has a first opening on a first side of the shank and a second opening on a second opposite side of the shank to allow the shank to flex in response to a load applied to the shank. The at least one slot can be positioned on a distal portion of the shank such that when the shank is advanced into a vertebra, the at least one slot is disposed in cancellous bone. The at least one slot can extend between proximal and distal ends of the shank to allow the shank to flex along the entire length of the shank. The shank of the bone screw can also be advanced over a guidewire. In one embodiment, the shank has a first stiffness zone that is slot-free and that is disposed in cortical bone, and a second stiffness zone having the at least one slot formed therein and disposed in cancellous bone. In another embodiment, the at least one slot includes a plurality of slots that allow the shank to flex in response to a load applied to the shank.

### BRIEF DESCRIPTION OF THE DRAWINGS

The invention will be more fully understood from the following detailed description taken in conjunction with the accompanying drawings, in which:

FIG. 1A is a perspective view of a bone screw having a shank with a slot-free proximal portion and a distal portion with a plurality of slots formed therein;

FIG. 1B is an enlarged view of the bone screw of FIG. 1A;

FIG. 2 is a side view of another embodiment of a bone screw having a shank with a plurality of proximal slots and a plurality of distal slots;

FIG. 3 is a side view of an embodiment of a bone screw having radially offset proximal and distal slots;

FIG. 4 is a perspective view of a bone screw having a shank with longitudinal slots, according to yet another embodiment;

FIG. 5 is a side view of another embodiment of a bone screw having a shank with a longitudinal slot that extends through the inner axis of the shank;

FIG. 6 a perspective view of a plurality of bone screws implanted in adjacent vertebral bodies;

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides bone screws and methods for accommodating different flexibility/stiffness regions in bone, and in particular in the pedicle of a vertebra. In an exemplary embodiment, various bone screws are provided having varying flexible regions. For example, the bone screw can have a first flexible region with a flexibility that corresponds to cortical bone, and a second flexible region with a flexibility that corresponds to cancellous bone. The different regions can be configured such that, when the bone screw is implanted, stiffer portions of the bone screw are implanted within the stiffer cortical bone and flexible and less stiff portions of the bone screw are implanted within the softer cancellous bone. Such a configuration will allow the bone screw to mimic the flexibility of the bone and thus move in coordination with the bone, thereby reducing the risk of fracture and/or loosening of the bone screw.

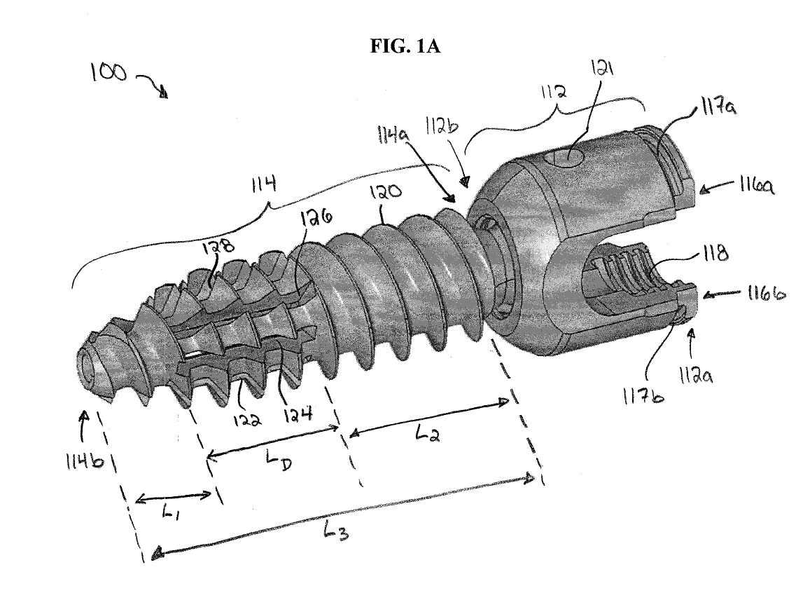

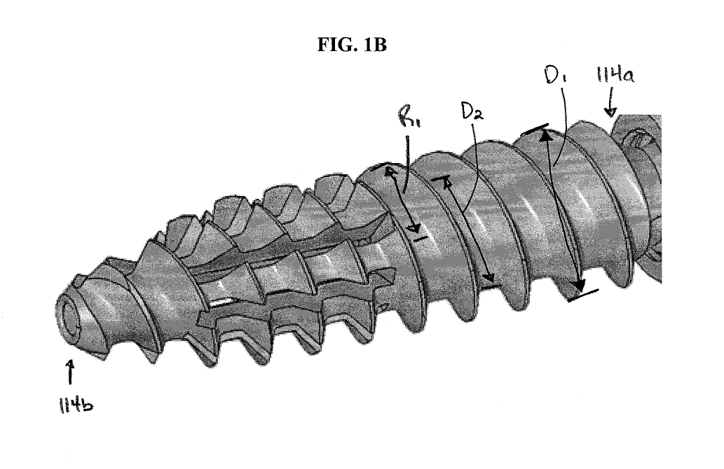

FIGS. 1A and 1B illustrate one exemplary embodiment of a bone screw 100. As shown in FIG. 1A, the bone screw 100 generally includes a U-shaped receiver member 112 for receiving a spinal fixation element, such as a spinal rod, and a threaded elongate shank 114 for engaging bone. The elongate shank 114 and the receiver member 112 can be joined in a variety of ways. For example, the elongate shank 114 can be fixedly mated to the receiver member 112, or as shown in FIGS. 1A and 1B the elongate shank 114 can be polyaxially coupled to the receiver member 112 to allow angular movement of the receiver member 112 relative to the elongate shank 114. In the illustrated embodiment, the receiver member 112 has an open proximal end 112a for receiving a fixation element and a substantially closed distal end 112b with a cavity formed therein that seats a head (not shown) formed on the proximal end 114a of the elongate shank 114.

While the receiver member can have a variety of configurations, in the illustrated embodiment the receiver member 112 has opposed side arms 116a, 116b that extend proximal from a substantially closed distal base, and that are substantially parallel to one another. The opposed side arms 116a, 116b define a U-shaped channel therebetween for seating a spinal fixation element. One skilled in the art will appreciate that the receiver member can be configured to receive a variety of fixation elements. Suitable spinal fixation elements for use with the present invention include, by way of non-limiting examples, rods, tethers, cables, plates, etc. The spinal fixation elements can have a variety of configurations, and, by way of non-limiting example, can be rigid, semi-rigid, bendable, flexible, etc. The distal end 112b of the receiver member 112 can have a concave cavity formed therein for polyaxially seating a head on the shank 114, and it can include an opening formed therethrough for receiving the shank 114.

The receiver member 112 can also include features to facilitate mating with various instruments used for implanting the receiver member 112, for positioning a spinal fixation element within the receiver member 112, and/or for mating a closure mechanism to the receiver member 112. For example, the receiver member 112 can include features for mating with a closure mechanism. As shown, an internal surface of each arm 116a, 116b can include one or more surface features formed thereon for mating with a closure mechanism. In the illustrated embodiment, each arm 116a, 116b has threads 118 formed on an internal surface thereof adjacent to the proximal end 112a of the receiver member 112. The threads allow a threaded closure mechanism, such as a set screw (not shown), to be threaded into the receiver member 112 to lock a spinal fixation rod therein.

The receiver member 112 can include a compression cap (not shown) disposed therein and configured to be positioned between the head on the proximal end 114a of the shank 114, and a spinal fixation element disposed within the receiver member 112. The compression cap can allow free polyaxial movement of the receiver member 112 relative to the shank 114 when a spinal fixation element is disposed within the receiver member 112, and the compression cap can be configured to lock the shank 114 in a fixed orientation relative to the receiver 112 when a closure mechanism is applied to the receiver member 112 to lock the spinal fixation element relative to the receiver 112. The receiver member 112 can also include features to retain and/or lock the compression cap therein. For example, the outer surface of each arm 116a, 116b can also include opposed bores (only one bore 121 is shown) formed therein, and an inner sidewall of each arm 116a, 116b can be deformable such that, when a pin or other member is inserted into each bore 121, the deformable portion swages inward to prevent proximal movement of a compression cap disposed within the receiver member 112. The bores 121 can be positioned at any location on the receiver member 112. In the illustrated embodiment, the bores 121 are positioned at a mid-portion of each arm 116a, 116b. The bores 121 can also or alternatively be configured to be engaged by an instrument, such as a rod reduction device or grasping device.

As further shown, the receiver member 112 can also include a groove 117a, 117b formed in an external surface of each arm 116a, 116b at a proximal end 112a of the receiver member 112. The grooves 117a, 117b can define flanges at the proximal end, which can facilitate grasping of the receiver member 112. For example, an extension cannula, rod reduction device, or other instrument can removably engage the grooves 117a, 117b to facilitate implantation of the bone screw 100, reduction of a spinal fixation element into the receiver member 112, and/or insertion of the closure mechanism into the receiver member 112. A person skilled in the art will appreciate that the receiver member 112 can include a variety of other features known in the art.

The elongate shank 114 can also have a variety of configurations and it can be formed from a variety of different materials. As shown in FIG. 1, the elongate shank 114 has a proximal end 114a and a distal end 114b, and includes threads 120 formed on an outer surface thereof for engaging bone. As discussed above, the proximal end 114a of the shank 114 can have a head (not shown) formed thereon that sits within a distal cavity formed within the receiver member 112, such that the shank 114 extends through the opening formed in the distal end 112b of the receiver member 112. Alternatively, the proximal end 114a of the shank 114 can be fixedly mated to a distal end 112b of the receiver member 112.

The size of the shank 114 and the threads 120 can vary depending on the intended use. For example, the minor diameter of the shank can remain constant along the entire length of the shank 114, between the proximal and distal ends 114a, 114b, or the minor diameter can decrease in a proximal-to-distal direction, as shown. The major diameter of the shank 114, i.e., the diameter of the threads 120, can also vary, and can remain constant or can likewise taper. The distal tip of the shank 114 can also have a variety of configurations, and it can be self-tapping. The shank 114 can also be cannulated for advancing the shank over a guidewire, as shown, or it can be non-cannulated. In certain exemplary embodiments, the shank 114 can have a length in the range of about 8 mm to 150 mm, a thread diameter D₁, also known as the major diameter, in the range of about 3 mm to 12 mm, and a root diameter D₂, also known as the minor diameter, in the range of about 2.5 mm to 10 mm. The thread pitch, or number of threads per unit length, can also vary, and in one embodiment the thread pitch can be in the range of about 1 mm to 4 mm. The elongate shank 114 can also be formed from various biocompatible materials including, by way of non-limiting example, surgical grade titanium, surgical grade stainless steel, cobalt chromium, and nitinol.

As further shown in FIGS. 1A and 1B, the shank 114 can include one or more slots formed therein and configured to provide varying regions of flexibility on the shank 114. As will be appreciated by a person skilled in the art, the elongate shank can include any number of slots positioned at various locations on the shank to form any number of stiffness regions, and the slots can be rectangular, oblong, or any other shape as may be desired to achieve the intended results. The slots can also have a depth less than the radius R₁ of the shank or they can have a depth greater than the radius R₁ and ranging up to a depth equal to the diameter D₁ of the shank such that the slot extends through the inner axis of the shank. In use, the slots will allow the shank to flex when the screw is implanted in bone.

In an exemplary embodiment, the shank includes regions of varying flexibility that correspond to the flexibility of the regions of bone within which the shank is intended to be implanted. A person skilled in the art will appreciate that the shank can include any number of flexibility regions, e.g., a single region, two regions, three regions, four regions, etc., as well as any number of slots in each region, e.g., 0, 1, 2, 3, 4, 5, 6, 7, 8, etc. slots in each region. Moreover, the flexibility regions can be positioned at any location along the length of the shank, and each region can be identical or can differ from one another.

By way of non-limiting example, as shown in FIGS. 1A and 1B, the elongate shank has a non-slotted proximal portion and a slotted distal portion which forms a single flexibility region. More particularly, at least one slot can be formed in the distal portion of the elongate shank and can extend along half or less than half of the length of the elongate shank. While the quantity of slots in each region can vary, in the illustrated embodiment the distal portion has eight separate and discrete slots (slots 122, 123, 126, 128 are shown) formed therein and spaced circumferentially around the shank 114. A person skilled in the art will appreciate that the elongate shank can include any number of distal slots and that the slots can be positioned symmetrically or asymmetrically about the elongate shank. In use, the slotted distal portion will be more flexible than the non-slotted proximal portion, which can be desirable when the bone screw will be implanted in a pedicle. In particular, when the bone screw 100 is implanted, the slotted distal portion can be configured to be disposed within the softer cancellous bone, while the slot-free proximal portion can be configured to be disposed within the harder cortical bone. Thus, as forces are applied to the receiver member 112, and thus to the shank 114, the shank 114 will flex in coordination with the bone. In other words, the slotted distal portion can flex in coordination with movement of the cancellous bone, while the slot-free proximal portion will have limited movement in coordination with the cortical bone and with the pedicle. Such a configuration can help prevent damage to the bone by the bone screw 100, as well as loosening or back-out of the bone screw 100.

While the particular dimensions can vary, by way of non-limiting example, the length of each distal slot L_{D} can be in the range of about 2 mm to 75 mm, and the length of the slot-free proximal portion L₂ can be in the range of about 2 mm to 75 mm. As a result, the ratio of the length of each distal slot L_{D} to the length of the shank L₃ can be in the range of about 0.1 to 0.75. As further shown in FIGS. 1A and 1B, the slots can also terminate just proximal to the distal tip 114b, such that the distal tip 114b is non-slotted to prevent the tip 114b from splaying as the elongate shank 114 is inserted into bone. The depth of the slots can also vary depending on the desired flexibility. The slots shown in FIGS. 1A and 1B have a depth that is substantially equal to (or slightly less than) the radius R₁ of the elongate shank such that the slots are in communication with the lumen extending through the cannulated shank 114. As a result, each slot is in direct communication with an opposing slot such that the shank 144 essentially includes four slots extending all the way through the shank 114 and intersecting one another. A distance L₁ between the distal tip 114b of the shank 114 and the terminal-most end of each slot can vary, for example the distance L₁ can be in the range of about 1 mm to 15 mm. Preferably, the distance is at least 4 mm so that the slots do not interfere with the non-slotted distal tip 114b. A person skilled in the art will appreciate that each slot can also vary with respect to one another, and that the particular dimensions and shape of each slot need not be identical.

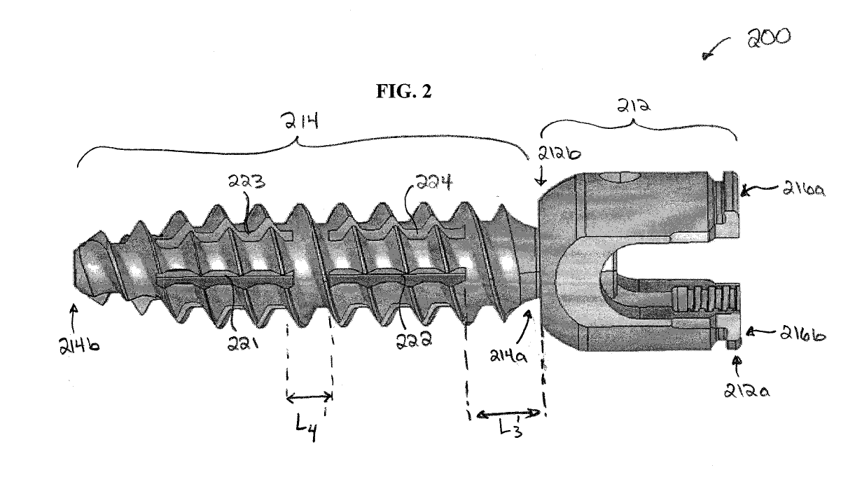

All of the features and dimensions discussed above with respect to FIGS. 1A and 1B apply equally to the embodiments shown in FIGS. 2-6. Except as otherwise discussed herein, like reference numerals are used to refer to corresponding parts, however the bone screws shown in FIGS. 2-6 have a different prefix (other than “1”) added to the reference numeral.

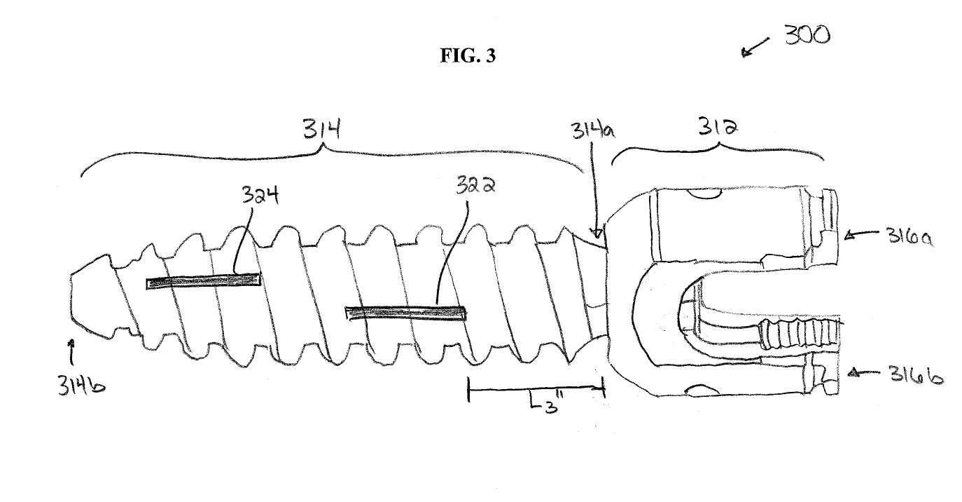

In another embodiment, the elongate shank can have slots on both the proximal and distal portions. For example, FIG. 2 illustrates an elongate shank 214 that includes at least one slot 222 in the distal portion 214b of the shank 214 and at least one slot 224 in the proximal portion 214a of the shank. As shown, the slots can be non-continuous and can form discrete flexibility regions on the elongate shank 214 that can correspond to flexibility regions in the type of bone that the bone screw will be implanted in. As will be appreciated by a person skilled in the art, the elongate shank 214 can include any number of slots on both the proximal and distal portions, and the slots can have a depth that is less than the radius R₁ of the elongate shank or greater than the radius R₁ and as large as the diameter D₁ of the elongate shank. In the embodiment shown in FIG. 2, the proximal and distal regions each include six discrete slots formed therein and spaced substantially equidistant from one another about a circumference of the shank 214. The proximal and distal slots are also longitudinally aligned, however they can alternatively be offset from one another. This is illustrated, for example, in FIG. 3, which shows a proximal slot 322 and a distal slot 324 longitudinally offset from one another. Continuing to refer to FIG. 2, the proximal slots can also optionally differ in size, quantity, and shape with respect to the distal slots. Such a configuration can allow the proximal slots to provide a first flexibility region that is different than a second flexibility region provided by the distal slots. Each slot in the proximal region and/or the distal region can also vary with respect to one another. As in the previous embodiment, a distal-most tip 314b of the shank 314 can be slot free. The proximal-most portion of the shank 314 can also be slot free. For example, a distance L₃′ between the distal end 212b of the receiver member 212 and the proximal-most end of the proximal slots 224 can be in the range of about 2 mm to 30 mm. The proximal and distal slots can also be spaced a distance apart from one another, as shown, to form a slot-free region L₄′ therebetween.

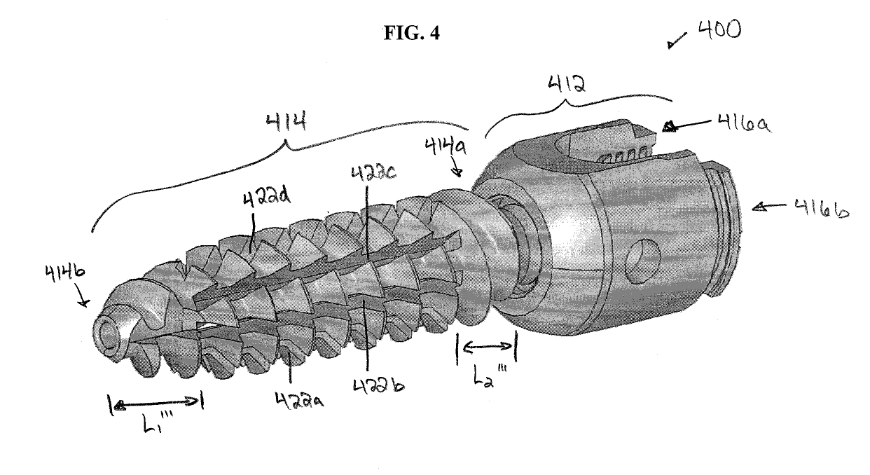

In another embodiment, the elongate shank can have a longitudinal slot that extends along a substantial length of the shank, i.e., from adjacent the proximal end to a location adjacent the distal end of the shank. In one embodiment, the slots can have a length that extends along about 30% to 95% of the length of shank 414 (as measured from the distal-most tip to the proximal-most end where the threads terminate). As shown in FIG. 4, the shank 414 includes eight slots positioned equidistance around a circumference thereof, with opposed slots being in communication with one another such that the shank 414 essentially includes four slots 422a, 422b, 422c, 422d extending therethrough and all intersecting one another. As a result, the shank 414 is cannulated to allow a guidewire to be received therein. The distal end in other embodiments, however, can be closed to form a non-cannulated shank 414. As in the previous embodiments, the elongate shank 414 can include any number of longitudinal slots positioned symmetrically or asymmetrically about the shank depending on the stiffness desired. The length of the slots 422a-d can also vary. In an exemplary embodiment, the shank 414 has a non-slotted portion L₂″′ between the proximal end 114a of the shank 114 and the proximal end of the slots, as well as a non-slotted portion between the distal end of the slots and the distal end 414b of the shank 414. The non-slotted proximal portion L₂″′ of the shank can be stiff and can be configured to be implanted in cortical bone, and the non-slotted distal portion L₁″′ can be stiff to prevent spreading of the distal tip 414b.

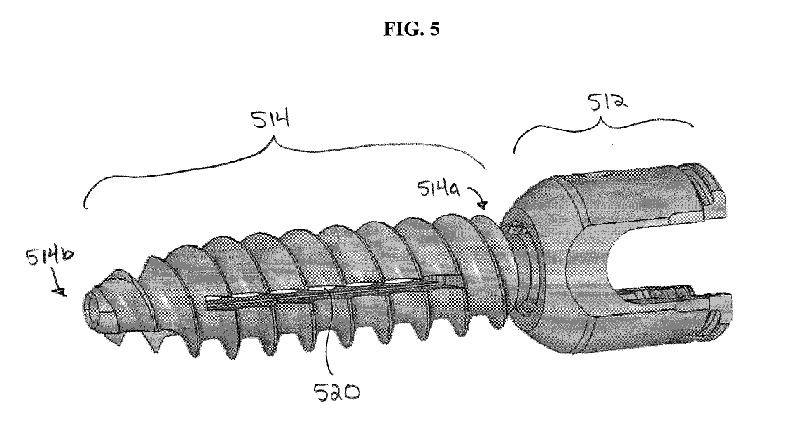

FIG. 5 illustrates yet another embodiment of a bone screw 500 having a shank with a slot formed therein. In this embodiment, the shank 514 has a single slot 520 formed therein and extending along a substantial portion of a length of the shank 514. As with the previous embodiment, the proximal end of the slot terminates at a distance distal to the proximal end 514a of the shank 514 to form a slot-free proximal portion, and the distal end of the slot terminates at a distance proximal to the distal end 514b of the shank 514 to form a slot-free distal tip. As further shown, the slot extends through the longitudinal axis of the shank and has a depth that is equal to or greater than the minor diameter of the shank. As a result, the shank 514 includes opposed slots formed therein. The illustrated shank 514 is cannulated, however the shank can alternatively be non-cannulated.

Although the illustrated bone screws have slotted shanks, the shank can also be formed from materials specifically selected to vary the shank flexibility. For example, a distal portion of a shank can be formed from a flexible material while a proximal portion of the shank can be formed from stiff material. The different materials can be joined in the manufacturing process in accordance with techniques known by a person skilled in the art, such as joints of composite polymer to metal. As will also be appreciated by a person skilled in the art, the elongate shank can be formed from different materials and can include any combination of longitudinal slots, proximal slots, and/or distal slots, having any combination of slot depths to alter the flexibility of the shank.

The illustrated bone screws can be used to stabilize a variety of bone structures, including by way of non limiting example, vertebral bodies. Where the desired use of the bone screw is for implantation in a vertebra, the first step is positioning and driving the screw to the desired depth in the vertebra. When the bone screw is cannulated, a guidewire can be used to position the bone screw. The cannulated bone screw can be advanced over the guidewire, which allows placement of the bone screw at a desired depth in bone. A pre-drilled hole can optionally be formed prior to advancing the bone screw over the guidewire. When the bone screw is non-cannulated, the screw is preferably inserted into a hole that is pre-drilled in bone using a drilling tool. Both cannulated and non-cannulated screws can be self-tapping to allow the screw to drive through bone. A person skilled in the art will appreciate that various techniques known in the art can be used to implant the bone screw in bone.

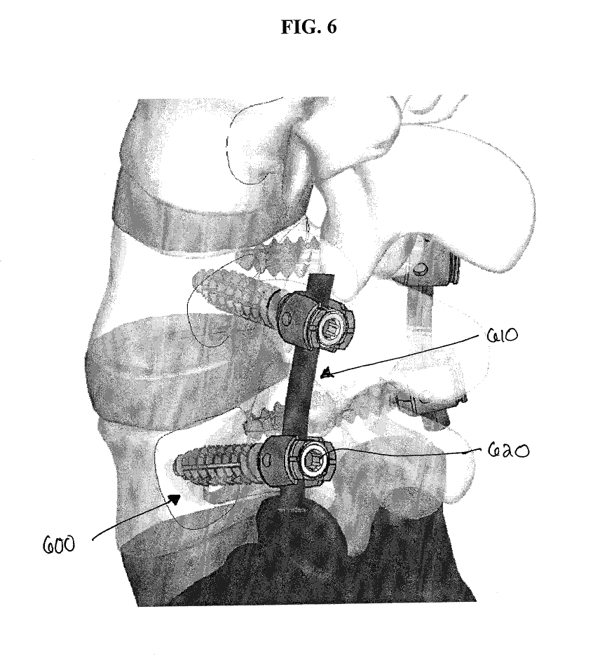

When two bone screws are fixed in adjacent vertebra, a spinal fixation element can be inserted into the receiver member of each screw. It can be difficult to position the spinal fixation element within each receiver member because of the alignment of the bone screws and the dimensions of the surgical site. As a result, a rod approximator device can be used to place the fixation element in the receiver members. The arms of the rod approximator device can include grasping members that fit into the corresponding recesses on the receiver member of the bone screw, stabilizing the rod approximator relative to the bone screw. With the rod pusher member in a first, proximal position, the device can be manipulated to place the spinal rod between a rod engaging member and the receiver member. The rod approximator can also include first and second handle members that can be grasped and squeezed together to cause the rod pusher member to move to a second, distal position, thereby causing the rod engaging member to grasp and push the fixation rod into the receiver member of the bone screw. After the rod is advanced into the receiver member, a closure mechanism can be applied to the receiver member of the bone screw to secure the rod. A fixation system is shown by way of non-limiting example in FIG. 6, including a bone screw 600, a fixation rod 610, and a closure mechanism 620. Once implanted, the slotted regions of the shank of bone screw are preferably disposed within cancellous bone, while the slot-free regions of the shank are preferably disposed within the harder, outer cortical bone. The slots remain open and are not filled with any materials to allow the bone screw to flex during movement of the adjacent vertebrae, and in response to any load applied thereto. The varying flexibility in the shank of the bone screw will reduce the stiffness of the screw shank, allowing a more smooth transfer of the load from the screw shank to the vertebra. In certain embodiments, depending on the configuration of the shank and on the implant location of the bone screw, the flexibility along the length of the shank can mimic the flexibility of the bone within which the shank is implanted.

Although the bone screws can be used in a pedicle, a person skilled in the art will appreciate that the bone screws disclosed herein can be used in all types of human skeletal structures. This includes, by way of non-limiting examples, vertebra, femur, tibia, hip, and skull.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A bone screw, comprising:
a receiver member having opposed arms configured to receive a spinal fixation element therebetween;
an elongate shank extending distally from the receiver member and having threads formed on an outer surface thereof for engaging bone, the elongate shank being non-cannulated and having at least one slot formed therein and extending in a proximal-distal direction, the at least one slot being configured to allow flexion of at least a portion of the elongate shank in response to a load applied to the shank when the shank is implanted in bone.

2. The bone screw of claim 1, wherein the at least one slot comprises at least one proximal slot formed in a proximal portion of the shank, and at least one distal slot formed in a distal portion of the shank and positioned distal of the at least one proximal slot.

3. The bone screw of claim 1, wherein the at least one slot comprises at least one proximal slot longitudinally aligned and non-continuous with at least one distal slot.

4. The bone screw of claim 1, wherein the at least one slot comprises a plurality of slots positioned symmetrically about the shank.

5. The bone screw of claim 1, wherein the at least one slot has a length substantially equal to a proximal-distal length of the shank

6. The bone screw of claim 1, wherein the at least one slot extends in a proximal-distal direction and extends through a central axis of the elongate shank.

7. The bone screw of claim 6, wherein the at least one slot comprises a plurality of slots positioned symmetrically about the shank.

8. A bone screw, comprising:
a receiver member having opposed arms configured to receive a spinal fixation element therebetween;
an elongate shank extending distally from the receiver member and having threads formed on an outer surface thereof for engaging bone, the elongate shank having at least one slot formed therein and extending in a proximal-distal direction, the at least one slot being selectively positioned such that, when the elongate shank is disposed within a pedicle of a vertebra, the shank has a flexibility that is configured to mimic a flexibility of the pedicle.

9. The bone screw of claim 8, wherein the at least one slot is positioned to form a first stiffness zone configured to mimic the flexibility of cortical bone and a second stiffness zone configured to mimic the flexibility of cancellous bone.

10. The bone screw of claim 8, wherein the at least one slot comprises a plurality of slots positioned symmetrically about the shank.

11. The bone screw of claim 8, wherein the at least one slot is positioned in the second stiffness zone, and the first stiffness zone is slot-free.

12. The bone screw of claim 8, wherein the at least one slot has a length substantially equal to a proximal-distal length of the shank.

13. The bone screw of claim 8, wherein the at least one slot comprises opposed openings in opposed outer surfaces of the shank, the opposed openings extending through a central axis of the shank.

14. A method for stabilizing bone structures, comprising:
advancing a shank of a bone screw into a vertebra such that threads on the shank threadably engage the vertebra, the shank having at least one slot extending in a proximal-distal direction;
positioning a spinal fixation rod in a rod receiver member coupled to a head on a proximal end of the shank of the bone screw; and
applying a fastening element to the rod receiver member to lock the spinal fixation rod relative to the rod receiver member;
wherein, after the fastening element is applied to the rod receiver member, the at least one slot allows the shank to flex in response to a load applied to the shank.

15. The method of claim 14, wherein the at least one slot extends through the shank such that the slot has a first opening on a first side of the shank and a second opening on a second opposite side of the shank to allow the shank to flex in response to a load applied to the shank.

16. The method of claim 14, wherein the at least one slot is positioned on a distal portion of the shank such that when the shank is advanced into a vertebra, the at least one slot is disposed in cancellous bone.

17. The method of claim 14, wherein the at least one slot extends between proximal and distal ends of the shank to allow the shank to flex along the entire length of the shank.

18. The method of claim 14, wherein the shank of the bone screw is advanced over a guidewire.

19. The method of claim 14, wherein the shank has a first stiffness zone that is slot-free and that is disposed in cortical bone, and a second stiffness zone having the at least one slot formed therein and disposed in cancellous bone.

20. The method of claim 19, wherein the at least one slot comprises a plurality of slots that allow the shank to flex in response to a load applied to the shank.
